(19) United States
(12) Patent Application Publication
(10) Pub. No.: US 2025/0268454 A1
(43) Pub. Date: Aug. 28, 2025

(54) SURGICAL ASSISTANCE SYSTEM AND SURGICAL ASSISTANCE DEVICE

(71) Applicants: National University Corporation Tokai National Higher Education and Research System, Nagoya-shi, Aichi (JP); Nagoya Denki Educational Foundation, Nagoya-shi, Aichi (JP)

(72) Inventors: Shota Nakamura, Nagoya-shi, Aichi (JP); Toyofumi YOSHIKAWA, Nagoya-shi, Aichi (JP); Takayuki KITASAKA, Toyota-shi, Aichi (JP); Yuichiro HAYASHI, Nagoya-shi, Aichi (JP); Kensaku MORI, Nagoya-shi, Aichi (JP)

(21) Appl. No.: 18/857,810

(22) PCT Filed: Mar. 8, 2023
(86) PCT No.: PCT/JP2023/008852

§ 371 Date: Oct. 17, 2024

(30) Foreign Application Priority Data
Apr. 18, 2022 (JP) 2022-068094

(51) Int. Cl. A61B 1/00; A61B 1/045; A61B 17/00; A61B 17/29; G06T 5/50; G06T 5/77; G06T 7/13; G06T 7/55; G06T 7/73; G16H 40/63

(52) U.S. Cl. CPC A61B 1/000095; A61B 1/000096; A61B 1/045; A61B 17/29; G06T 5/50; G06T 5/77; G06T 7/13; G06T 7/55; G06T 7/73; G16H 40/63; A61B 2017/00778; G06T 2207/10068; G06T 2207/20081; G06T 2207/20212; G06T 2207/30096

(57) ABSTRACT

An object is to provide a surgery support system that can ensure a better field. The object can be achieved by a surgery support system including a surgical assistance device, an image processing unit, and a display unit. The image capturing units are configured such that a plurality of image capturing units are provided to the surgical assistance device, the image capturing units captures an image so that inside of the body cavity appears in the image, and when the treatment tool is inserted in the body cavity, the image capturing units captures an image so that the inside of the body cavity including the treatment tool appears in the image, and the image processing unit is configured to synthesize images obtained from image capturing units to generate synthesized image, and perform an obstructing object removal process of removing at least a part of image of the treatment tool.

### TECHNICAL FIELD

The disclosure in the present application relates to a surgery support system and a surgery support device.

### BACKGROUND ART

In the field of surgical operations, endoscopic surgeries such as laparoscopic surgeries or thoracoscopic surgeries have rapidly become prevalent in place of surgeries under direct view such as conventional laparotomy surgeries or thoracotomy serveries. The endoscopic surgeries have various advantages in terms of esthetic outcome, low invasiveness, or the like.

In endoscopic surgeries, unlike laparotomy surgeries or thoracotomy serveries, the operator is unable to directly view the affected part. Thus, there is a known example in which a plurality of trocars having cameras are inserted into the body, images obtained from the cameras are synthesized based on camera positions estimated by position sensors, and the operator operates a surgical instrument such as a forceps while viewing a monitor displaying the synthesized image (see Patent Literature 1).

### CITATION LIST

### Patent Literature

Patent Literature 1: Japanese Patent No. 5975504

### Non-Patent Literature

Non-Patent Literature 1: Takayuki Kitasaka et. al., “A study on surgical field retrieval for intelligent laparotomy video archive system”, International Forum on Medical Imaging in Asia (IFMIA), pp. 327-328, 2017.

### SUMMARY OF INVENTION

### Technical Problem

Even with today's advanced medical technologies, deaths due to intraoperative vascular injuries have been reported. According to a questionnaire survey by the Japanese Society for Endoscopic Surgery, many surgeons cite “insufficient field of view” as one of the causes of vascular injuries. It is thus desired to ensure a wide field of view required for proceeding a safe surgery (hereafter, which may be simply referred to as “field of view”) in endoscopic surgeries. In the art disclosed in Patent Literature 1, however, each camera is arranged at the tip of the trocar inserted into the body. Therefore, although it is possible to capture an image of a part of the body inside the camera, it is not possible to capture an image of a part of the body outside the camera, and thus, there is a problem of inability of panoramically viewing the field of view.

Further, the art disclosed in Patent Literature 1 requires providing position markers to a plurality of trocars, detecting the position markers by position sensors, and synthesizing images obtained based on estimated positions of cameras. Thus, there is a problem of complex image synthesis.

To solve such a problem, the inventors of the present application have proposed an invention in International Application No. PCT/JP2021/39295 that enables a sufficient field of view in an endoscopic surgery to be ensured by arranging a surgical assistance device having three or more image capturing units arranged to a base member at an incision site without requiring position markers. However, since endoscopic surgeries are performed in a three-dimensional manner within a limited narrow range inside a body cavity, it is desirable to ensure a better field of view as much as possible.

That is, an object of the disclosure in the present application is to provide a surgery support system and a surgery support device that can ensure a better field of view during an endoscopic surgery.

### Solution to Problem

The disclosure in the present application relates to a surgery support system and a surgery support device illustrated below.
(1) A surgery support system comprising:
   a surgical assistance device configured to assist use of a treatment tool inserted into a body cavity and having image capturing units configured to capture images inside the body cavity;
   an image processing unit configured to process images obtained from the image capturing units; and
   a display unit configured to display image data image-processed by the image processing unit,
   wherein the image capturing units are configured such that
   a plurality of image capturing units are provided to the surgical assistance device,
   each of the image capturing units captures an image so that inside of the body cavity appears in the image, and
   when the treatment tool is inserted in the body cavity, any of the image capturing units captures an image so that the inside of the body cavity including the treatment tool appears in the image, and
   wherein the image processing unit is configured to
   synthesize images obtained from respective image capturing units to generate a synthesized image, and
   perform an obstructing object removal process of removing at least a part of an image of the treatment tool.
(2) The surgery support system according to (1) above,
   wherein the treatment tool has
   a treatment part used for treatment inside the body cavity, and
   a non-treatment part continuous to the treatment part, and
   wherein the image processing unit generates an obstructing object removed image by combining at least
   a position indication image indicating a position of the non-treatment part based on the shape of the non-treatment part, and
   a hidden-part image that is an image hidden by the non-treatment part.
(3) The surgery support system according to (2) above, wherein the image processing unit generates the position indication image by performing machine learning on the non-treatment part from training images of the treatment tool.
(4) The surgery support system according to (3) above, wherein the position indication image is an image indicating a contour of the non-treatment part.
(5) The surgery support system according to any one of (1) to (4) above further comprising an autofocus unit configured to adjust focal distances of the image capturing units individually.
(6) The surgery support system according to any one of (1) to (4) above further comprising a depth estimation unit configured to estimate a depth to a point of interest inside the body cavity.
(7) A surgery support device comprising: an image processing unit configured to process images obtained from a plurality of image capturing units of a surgical assistance device configured to assist use of a treatment tool inserted into a body cavity,
   wherein the surgery support device
   synthesizes the images obtained from respective image capturing units to generate a synthesized image, and
   performs an obstructing object removal process of removing at least a part of an image of the treatment tool.
(8) A program used in the surgery support system according to any one of (1) to (4) above or the surgery support device according to (7) above.

### Advantageous Effects of Invention

The surgery support system and the surgery support device disclosed in the present application can be suitably used for endoscopic surgeries.

### BRIEF DESCRIPTION OF DRAWINGS

FIG. 1 is a configuration diagram of a surgery support system according to an embodiment.

FIG. 2A is a schematic top view of a surgical assistance device 1, FIG. 2B is a sectional view taken along X-X′ of FIG. 2A, and FIG. 2C is a diagram with removal of an image capturing unit 2 from FIG. 2B.

FIG. 3 is a block diagram schematically illustrating a hardware configuration of an image processing unit 14.

FIG. 4 is an explanatory diagram schematically illustrating a relationship between the surgical assistance device 1, a forceps 22, an organ 24, and captured images 32-1 to 32-3 taken by respective image capturing units 2.

FIG. 5 is an explanatory diagram schematically illustrating a relationship between the captured images 32-1 to 32-3 and a synthesized image 34.

FIGS. 6(a) to (d) are explanatory diagrams sequentially illustrating steps of an obstructing object removal process related to the overall forceps 22.

FIG. 7 is an explanatory diagram schematically illustrating an image of the forceps 22 after the obstructing object removal process related to a handle 22B has been performed.

FIG. 8 is an explanatory diagram illustrating an image cut out from a simulation moving image for a surgery.

FIG. 9 is an explanatory diagram illustrating a cause of occurrence of out-of-focus in a synthesized image.

FIG. 10(a) is an explanatory diagram illustrating an example of an image before calibration, and FIG. 10(b) is an explanatory diagram illustrating an example of an image after calibration.

FIG. 11(a) is an explanatory diagram illustrating an example of a synthesized image before autofocusing, and FIG. 11(b) is an explanatory diagram illustrating an example of a synthesized image after autofocusing.

### DESCRIPTION OF EMBODIMENTS

### Overview of Surgery Support System 10

A surgery support system and a surgery support device disclosed in the present application will be described below in detail. Note that the position, size, range, or the like of each component illustrated in the drawings may not represent the actual position, size, range, or the like for easier understanding. Thus, the disclosure of the present application is not necessarily limited to the position, the size, the range, or the like disclosed in the drawings.

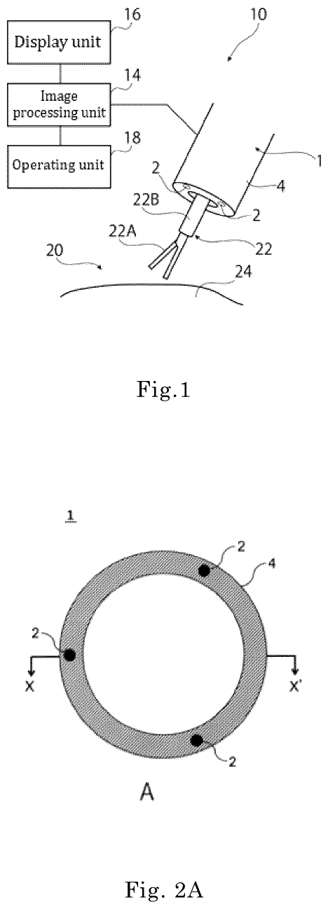

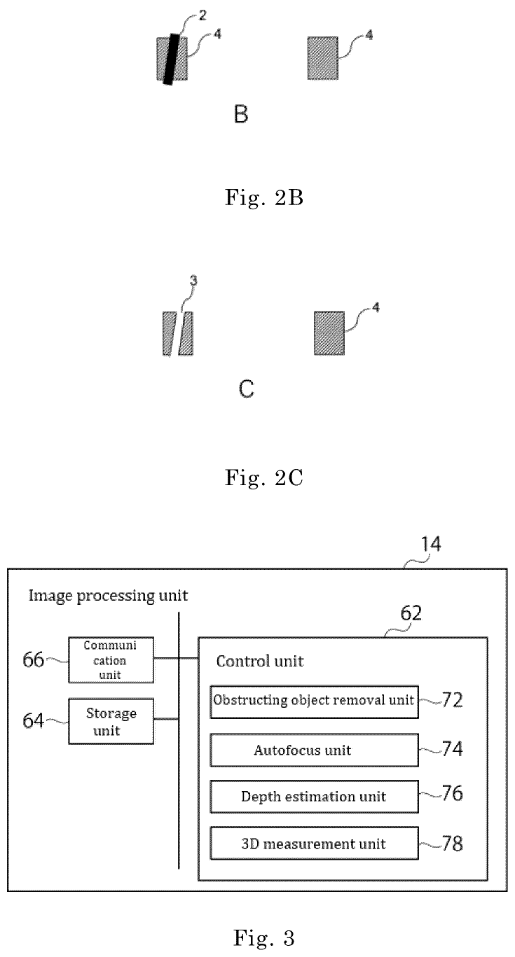

A surgery support system 10 according to the embodiment will be described with reference to FIG. 1 and FIG. 2. The surgery support system 10 includes a surgical assistance device 1, an image processing unit 14, a display unit 16, an operating unit 18, and the like as main features. The surgical assistance device 1 assists the use of a treatment tool (a forceps 22 in this example) inserted into a body cavity 20 and has image capturing units 2 that captures an image inside the body cavity 20 (FIG. 2A to FIG. 2C). The image processing unit 14 processes images obtained from the image capturing units 2. The display unit 16 displays image data image-processed by the image processing unit 14. The operating unit 18 is used for entry of information required for causing each instrument of the surgery support system 10 to exert the function thereof.

A plurality of image capturing units 2 are provided to the surgical assistance device 1, each image capturing unit 2 captures an image so that the inside of a body cavity appears in the image and, when a treatment tool is inserted in the body cavity, any of the image capturing units 2 captures an image so that the inside of the body cavity including the treatment tool appears in the image. The image processing unit 14 synthesizes images obtained from respective image capturing units 2 to generate a synthesized image and performs an obstructing object removal process of removing at least a part of the image of the treatment tool. The image processing to generate a synthesized image is image processing to synthesize moving images captured by a plurality of image capturing units 2 and thereby generate an image having a wider field of view than a field of view obtained by a single image capturing unit 2. The obstructing object removal process is performed by using a parallax difference between a plurality of image capturing units 2.

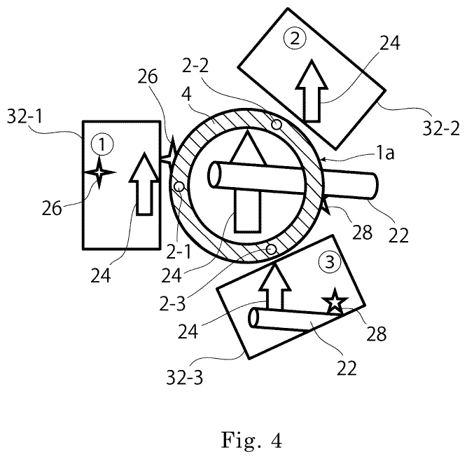

As illustrated in FIG. 1, the surgical assistance device 1 may be used as an insertion port that is for inserting a surgical treatment tool into the body cavity 20. In such a case, the surgical assistance device 1 is a trocar with a camera also having an image capturing function. Further, a treatment tool may be inserted into the body cavity 20 from a hole other than a hole opened for the surgical assistance device 1. In such a case, the surgical assistance device 1 captures an image of the treatment tool inserted from another hole. FIG. 4 schematically illustrates synthesis of images with a case where the surgical assistance device 1 is inserted from a hole other than a hole used for insertion of the treatment tool.

As the treatment tool, there are various instruments such as the forceps 22 used for surgeries. In general, there are various types of forceps 22 for respective functions, respective organs, or respective purposes, and any type thereof may be employed.

Note that the surgical assistance device 1 can be any device that assists the use of a treatment tool inserted in the body cavity 20 and may be, for example, a device used in such a way that the surgical assistance device 1 itself is inserted into a body cavity. Further, the surgical assistance device 1 may be a device used in such a way that the surgical assistance device 1 itself is not inserted into a body cavity but is installed at an incision site of the body.

The same surgical assistance device 1 as that disclosed in International Application No. PCT/JP2021/39295 by the present applicant can be used. The subjects disclosed in International Application No. PCT/JP2021/39295 are incorporated in the present specification by reference. FIG. 2A illustrates the surgical assistance device 1 similar to that disclosed as one embodiment in the same application. FIG. 2A is a schematic top view of the surgical assistance device 1, FIG. 2B is a sectional view taken along X-X′ of FIG. 2A, and FIG. 2C is a diagram with removal of the image capturing unit 2 from FIG. 2B.

The surgical assistance device 1 includes the image capturing units 2, holding parts 3 that hold the image capturing units 2, and a base member 4. In the example illustrated in FIG. 2A to FIG. 2C, the base member 4 includes the holding parts 3. The base member 4 is formed in a tube-like shape, and the image capturing units 2 are attached to the base member 4 through the holding parts 3. Although the image capturing units 2 and the holding parts 3 are built in the base member 4 in the example illustrated in FIG. 2A to FIG. 2C, the image capturing units 2 (and/or the holding parts 3) may protrude from the outer circumferential part of the base member 4.

Each image capturing unit 2 is not particularly limited as long as it can capture an image inside a body cavity. For example, a CCD image sensor, a CMOS image sensor, Foveon X3, an organic thin-film image capturing element, or the like may be employed. Note that the image capturing range of the image capturing unit 2 is not particularly limited. While it is conceivable to use a wide-angle camera to capture an image inside a body cavity by a single image capturing unit 2, in such a case, the edge of an image may be blurred. Further, there may be a part unable to be captured due to a surgical instrument, an organ, or the like (a part hidden behind the same). In contrast, since the surgical assistance device 1 is equipped with three or more image capturing units 2, it is possible to ensure a sufficient field of view and reduce a hidden part even with use of the sensor or the like described above that are commonly, commercially available.

In the example illustrated in FIG. 2A, three image capturing units 2 are provided to the base member 4. Although a sufficient field of view was not ensured in some cases when the number of image capturing units 2 is two, a sufficient field of view was ensured when the number thereof was three or greater. It is therefore preferable that the number (N) of image capturing units 2 be an integer of three or greater and may be, for example, four or greater, five or greater, six or greater, or the like. On the other hand, although there is no limitation on the upper limit of the number of image capturing units 2 in terms of ensuring a sufficient field of view, a larger number of image capturing units 2 will make the process in image synthesis complex and also increase the cost. Therefore, the upper limit of the number (N) of the image capturing units 2 can be considered taking the cost, the usability (processing speed) of image processing, or the like into account and may be, for example, 20 or less, 15 or less, 10 or less, 8 or less, or the like.

The holding parts 3 are provided in the base member 4 for holding the image capturing units 2. Although each holding part 3 is formed so as to penetrate through the base member 4 in the example illustrated in FIG. 2A to FIG. 2C, the shape or the arranged place of the holding part 3 is not particularly limited as long as it can hold the image capturing unit 2 at a predetermined angle. Each of the image capturing units 2 is arranged so as to be directed to outside of the base member 4 at an angle of 0 degree to 10 degrees.

Although depiction or detailed description will be omitted, the surgical assistance device 1 is provided with an endoscope or a light source. For the light source, for example, a chip LED is used, which is connected to an external power source. Moreover, a sealing mechanism that prevents leakage of air during insertion and extraction of a treatment tool (the forceps 22 in the present embodiment), an air supply mechanism that supplies air into an abdominal cavity, or the like can be provided to the surgical assistance device 1. The image capturing unit 2 has a zooming function or an autofocus function. The zooming function may be one relying on optical zooming or one relying on digital zooming. The autofocus function will be described later.

As illustrated in FIG. 1, the surgical assistance device 1 is connected to the image processing unit 14. Image data acquired by the image capturing units 2 are transmitted to the image processing unit 14. The connection system between the image capturing unit 2 and the image processing unit 14 may be wired or wireless.

The image capturing unit 2 captures an image including a treatment tool, and the treatment tool has a treatment part used for treatment inside the body cavity 20 and a non-treatment part continuous to the treatment part. When the treatment tool is the forceps 22, a pinch part 22A on the tip side corresponds to the treatment part, and a handle 22B continuous to the pinch part 22A corresponds to the non-treatment part. The pinch part 22A is formed with a material such as a stainless alloy, and the handle 22B is coated with an electrical insulating material. The pinch part 22A can also be referred to as “function part” or the like.

### Hardware of Image Processing Unit 14

The image processing unit 14 can be configured by installing a program (software) for causing a computer device to exert each function of the surgery support system 10. While the image processing unit 14 forms a surgery support device in the surgery support system 10, the entire surgery support system 10 can be referred to as a surgery support device. In such a case, the image processing unit 14 forms a part of the surgery support device.

As illustrated in FIG. 3, the computer device includes a control unit 62, a storage unit 64, and a communication unit 66. The control unit 62 has one or a plurality of processors and peripheral circuits thereof. The control unit 62 controls the overall operation of the image processing unit 14 in an integrated manner and is a central processing unit (CPU), for example. The control unit 62 performs processes based on programs (computer programs such as a driver program, an operating system program, an application program, or the like) stored in the storage unit 64. Further, the control unit 62 can execute a plurality of programs in parallel.

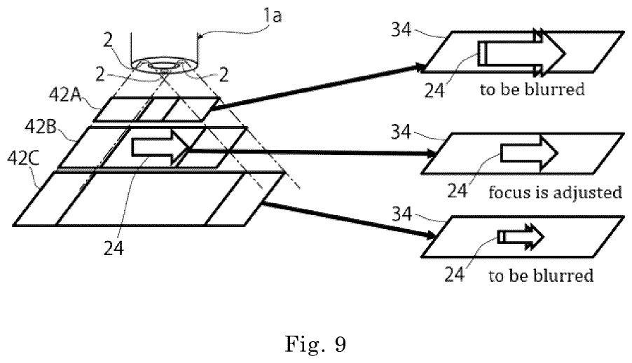

The control unit 62 includes an obstructing object removal unit 72, an autofocus unit 74, a depth estimation unit 76, and a 3D measurement unit 78. The obstructing object removal unit 72 performs an obstructing object removal function in accordance with an obstructing object removal process described later (FIG. 4 to FIG. 8). The autofocus unit 74 performs an autofocus function (FIG. 9 to FIG. 11). The depth estimation unit 76 performs a depth estimation function, and the 3D measurement unit 78 performs a 3D measurement function.

Each of these units in the control unit 62 is a function module implemented by a computer program executed on the processor of the control unit 62. Each of these units in the control unit 62 may be implemented in the image processing unit 14 as a separate integrated circuit, a microprocessor, or firmware.

The storage unit 64 is used for storing information required for performing each function by the control unit 62. For example, the storage unit 64 has at least one of a semiconductor memory, a magnetic disk device, and an optical disk device. The storage unit 64 stores a driver program, an operating system program, an application program (such as a control program for implementing the function of the image processing unit 14), data, or the like used for processes performed by the control unit 62. For example, the storage unit 64 stores a communication device driver program, which controls the communication unit 66 described later, or the like as the driver program.

The computer program may be installed to the storage unit 64 by using a known setup program or the like from a computer readable portable storage medium such as a CD-ROM, a DVD-ROM, or the like, for example. Further, the computer program may be a program downloaded from cloud via a public communication line such as the Internet communication line.

The communication unit 66 has an interface circuit used for performing wired communication according to a communication system such as Ethernet (registered trademark) or wireless communication according to a communication system such as Wi-Fi (registered trademark), Wi-Fi Aware (registered trademark), or the like and establishes wired communication or wireless communication with communication units (not illustrated) included in the surgical assistance device 1, the display unit 16, the operating unit 18, or other external instruments (not illustrated) or the like to directly perform transmission of information.

The interface circuit of the communication unit 66 may be near field communication according to a communication system such as Bluetooth (registered trademark), communication using 920 MHz-band specified low-power radio, or the like. The communication unit 66 is not limited to one for performing wireless communication and may be one for transmitting various signals by infrared communication or the like, for example. Further, the communication unit 66 may be a communication interface for connecting to a universal serial bus (USB) or the like, a communication interface for a wired or wireless local area network (LAN) or the like.

### Obstructing Object Removal Function

Next, the obstructing object removal function of the image processing unit 14 will be described. The image processing unit 14 generates an obstructing object removed image by using at least a position indication image, which indicates a position of a non-treatment part based on the shape of the non-treatment part (the handle 22B in this example), and a hidden-part image, which is an image hidden behind the non-treatment part. The obstructing object removed image is an image that can indicate the position or the region of the non-treatment part. The obstructing object removed image is generated by replacing an image of a non-treatment part (an image of the region hidden by the handle 22B in this example) with an image obtained from another image capturing unit 2.

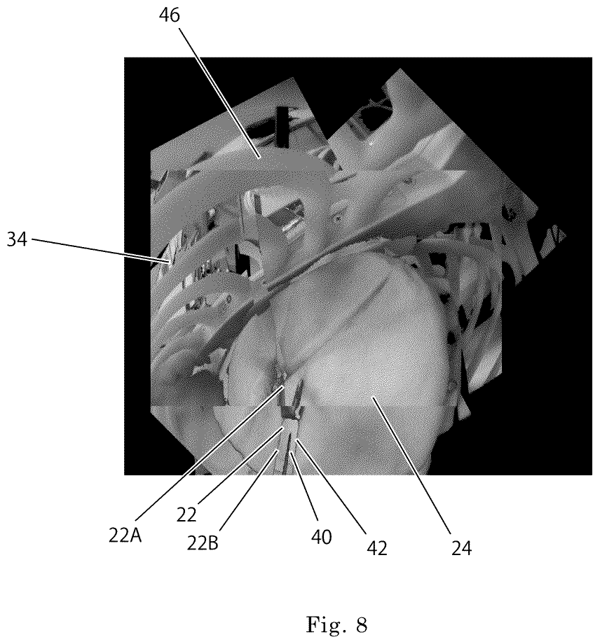

In the following, first, the obstructing object removal process to remove (also referred to as “delete”) the entire obstructing object will be described based on FIG. 4 to FIG. 6. Then, the obstructing object removal process to remove the obstructing object while leaving a part thereof will be described based on FIG. 7 and FIG. 8.

FIG. 4 schematically illustrates a process of generating a synthesized image from images acquired by the three image capturing units 2 and a process of removing an obstructing object from the synthesized image. In FIG. 4, the surgical assistance device 1 is illustrated at the center. In FIG. 4, an organ 24 in the body cavity 20 is simplified by a figure of an arrow, and the forceps 22 is simplified by a figure of a circular bar.

In FIG. 4, the tip of the surgical assistance device 1 is spaced apart from and faces the organ 24, and the tip portion of the forceps 22 has entered the area between the tip of the surgical assistance device 1 and the organ 24. Herein, FIG. 4 schematically illustrates a situation where the forceps 22 has been inserted in the body cavity 20 from a hole other than the hole opened for the surgical assistance device 1 rather than a situation where the surgical assistance device 1 is used as an insertion port for inserting the forceps 22 into the body cavity 20. In FIG. 4, the left side of the forceps 22 is the tip side (the side of the pinch part 22A), and the right side of the forceps 22 corresponds to the base end side (the side of the handle 22B).

In the present embodiment, the obstructing object is the forceps 22 or the handle 22B. The forceps 22 is located between each of the image capturing units 2-1 to 2-3 and the organ 24 and thus is an obstructing object that hides the organ 24 or the surgical operation site.

In FIG. 4, a branched number is added to each of the plurality of (in this example, three) image capturing units 2, and the references of the image capturing units 2 are “2-1”, “2-2”, and “2-3”. Moreover, in FIG. 4, respective images captured by the image capturing units 2-1 to 2-3 (also referred to as “captured image”) are labeled with references 32-1, 32-2, and 32-3, respectively. Note that, in FIG. 4, encircled numbers “1” to “3” are indicated for the captured images 32-1, 32-2, and 32-3. In FIG. 4, these numbers are illustrated so that the captured images 32-1, 32-2, and 32-3 can be clearly distinguished, and such encircled numbers are not superimposed on the actual images.

In FIG. 4, at least a part of the organ 24 appears in the captured images 32-1 to 32-3 from the image capturing units 2-1 to 2-3, respectively. While particular captured images 32-2 and 32-3 out of the captured images 32-1 to 32-3 include the forceps 22, the remaining captured image 32-1 does not include the forceps 22.

Those indicated by references 26, 28 in FIG. 4 are peripheral objects of the organ 24, and these peripheral objects 26, 28 are simplified by stars. One peripheral object 26 appears in the captured image 32-1, and the other peripheral object 28 appears in the captured image 32-3. The peripheral objects 26, 28 can be said as “organ adjacent” to the organ 24 targeted for incision or “organ present around” the organ 24 targeted for incision or the like.

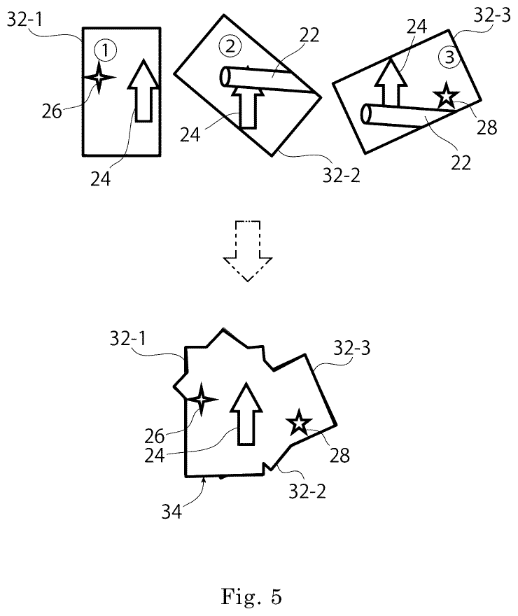

The captured images 32-1 to 32-3 are synthesized by the image processing unit 14 as schematically illustrated in FIG. 5. The captured images 32-1 to 32-3 before synthesized are arranged and illustrated in the upper part in FIG. 5, and the captured images 32-1 to 32-3 after synthesized are illustrated in the lower part in FIG. 5. In the following, a captured image after synthesized is referred to as “synthesized image”, and the synthesized image is labeled with reference 34.

The synthesized image 34 is generated by combination of the captured images 32-1 to 32-3 in accordance with the arrangement or inclination based on the positional relationship between the image capturing units 2-1 to 2-3. In the synthesized image 34, the organ 24 and the peripheral objects 26, 28 are displayed so as to match to the actual positional relationship. Respective captured images 32-1 to 32-3 are moving images, and the synthesized image 34 is also displayed as a moving image. The image processing unit 14 can also record the moving image of the synthesized image 34 or cut out the synthesized image 34 to acquire image data of a static image.

In the upper part in FIG. 5, the forceps 22 appears in the captured images 32-2 and 32-3. While the organ 24 and the peripheral objects 26, 28 appear in the synthesized image 34 in the lower part in FIG. 5, the forceps 22 does not appear. In this generation of the synthesized image 34, the image of the forceps 22 that appeared in the captured images 32-2 and 32-3 has been removed.

In the present embodiment, removal of the image of the forceps 22 is automatically performed when the synthesized image 34 is generated. However, the timing of removal of an image is arbitrary, and an image may be removed by the operator inputting an instruction to the image processing unit 14 via the operating unit 18, for example. Further, the image processing unit 14 may have a speech recognition function, and the removal of an image may be performed when a voice instruction is detected.

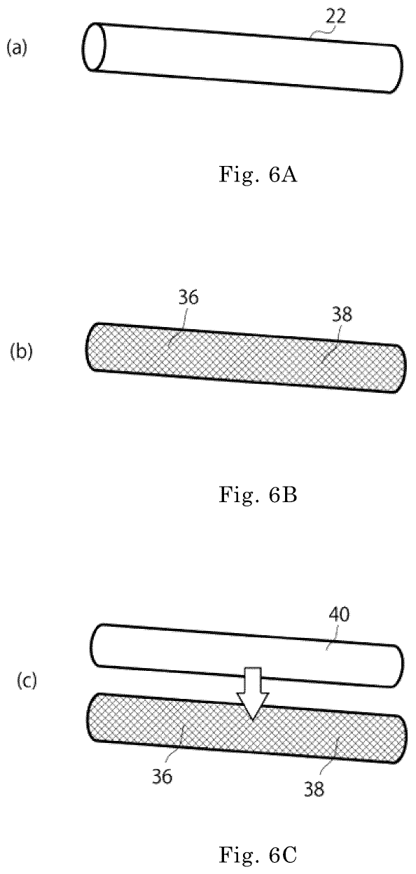

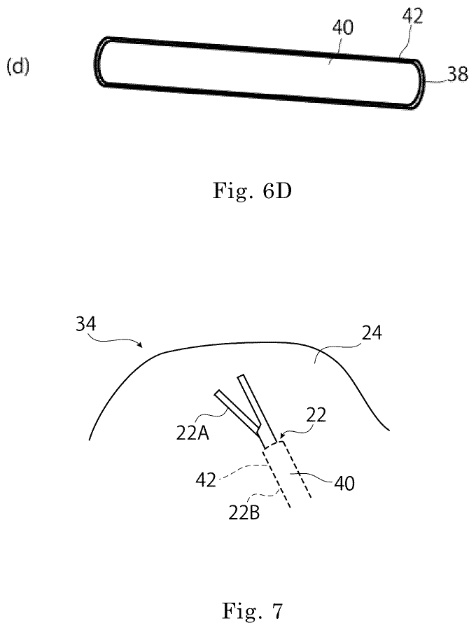

In removal of the image of the forceps 22, the image of the forceps 22 in the synthesized image 34 is detected, and a process to remove the detected image of the forceps 22 is performed. FIG. 6(a) to (d) schematically illustrate the procedure of image removal. In FIG. 6(a), the forceps 22 is illustrated in a simplified manner by a circular bar. In subsequent FIG. 6(b), the image of the forceps 22 has been removed. Further, in FIG. 6(b), a region (also referred to as “obstructing object removed region”) 36 in which the image of the forceps 22 was displayed in FIG. 6(a) is represented as an obstructing object removed image 38 in a form in only black, only gray, or the like, for example.

As illustrated in FIG. 6(c), a hidden-part image 40 is superimposed on the obstructing object removed image 38. The hidden-part image 40 is generated for supplementing the obstructing object removed region 36. Generation of the obstructing object removed region 36 will be described later.

In subsequent FIG. 6(d), a linear contour part 42 appears around the hidden-part image 40. The contour part 42 is a position indication image for the forceps 22. The position indication image is an image indicating a region of a treatment tool (the forceps 22 in this example), as the position of the treatment tool, to the surgical operator or the like viewing the display unit 16. The reason why the contour part 42 appears is that a slight difference in sizes or a misalignment of positions between the obstructing object removed image 38 and the hidden-part image 40 is expressed as a difference.

As illustrated in FIG. 6(b), when the entire forceps 22 is removed, the image of the forceps 22 is detected. Detection of the image of the forceps 22 is performed by using results of machine learning performed in advance. The image processing unit 14 performs machine learning on the treatment tool from training images (also referred to as “training data”) of the treatment tool (the forceps 22 in this example) to generate a position indication image (an image of the contour part 42).

For the machine learning, a number of (for example, several thousands) image data related to the forceps 22 are accumulated in advance. Although accumulation of image data for the machine learning is performed by using the storage unit 64 (FIG. 3) of the image processing unit 14 in the present embodiment, the accumulation may be performed by using a storage unit outside the image processing unit 14 (including a storage unit on cloud).

The training data are acquired via image capturing of the forceps 22 at various conceivable directions or angles. Moreover, detection of images in which the pinch part 22A and the handle 22B are distinguished from each other is performed for each image of the training data. In the image processing unit 14, the regions of the forceps 22, the pinch part 22A, and the handle 22B are distinguished by using results of image detection.

The pinch part 22A and the handle 22B of the forceps 22 are distinguished from each other based on results of determination on the shape or the color thereof. The color of the pinch part 22A is, in general, silver that is the color of the raw material of stainless alloys (also referred to as “silver color”).

The handle 22B is, in general, covered with a cover (not illustrated), and electrically insulating synthetic resins or the like are used for the material of such a cover. Further, in general, colors that enable distinction from stainless steel, such as black or brown, are employed for the color of such a cover. Such a difference in partial coloring (color scheme) in the forceps 22 is used, and thereby the pinch part 22A and the handle 22B are distinguished from each other in an image.

Although the pinch part 22A and the handle 22B can also be distinguished from each other by using only the information on colors, use of information on shapes in combination can reduce the impact of noise, and higher detection accuracy is obtained. Further, it is theoretically possible to distinguish the pinch part 22A and the handle 22B from each other by using only the information on shapes. However, the detection accuracy is higher when the information on colors is used.

In such a way, by distinguishing the forceps 22 for each portion, it is possible to perform removal of the image of the forceps 22 not only on the entire forceps 22 but also on a part of the forceps 22 (the handle 22B in this example), as illustrated in FIG. 7 and FIG. 8 described later.

As illustrated in FIG. 6(b), when the image of the entire forceps 22 is removed, the image processing unit 14 sequentially compares the image related to the entire forceps 22 in the synthesized image 34 with an image related to the entire forceps 22 in the training data. The image processing unit 14 recognizes a region related to the entire forceps 22. Moreover, the image processing unit 14 removes the image of the forceps 22 from the synthesized image 34.

The removed region is a portion hidden behind the forceps 22. Synthesis of the hidden-part image 40 has been performed on the hidden part (FIG. 6(c), (d)). Data on the hidden-part image 40 (hidden-part image data) is created via selective use of the captured images 32-1 to 32-3 from the image capturing units 2-1 to 2-3. For the hidden-part image 40, an image of a portion corresponding to a region inside the contour of the forceps 22 is used out of the captured image not including the forceps 22 (the captured image 32-1 in the example of FIG. 4 and FIG. 5).

The hidden-part image 40 is synthesized to the removed portion of the synthesized image 30, and thereby the synthesized image 30 from which the forceps 22 has been removed is generated (FIG. 5). In FIG. 5, illustration of the hidden-part image 40 or the contour part 42 is omitted.

For example, the contour part 42 illustrated in FIG. 6(d) is not such a part that is displayed with an image of a line being superimposed on the portion of the contour of the hidden-part image 40. Further, in many cases, the forceps 22 is not stationary but is moving. Thus, the contour part 42 is not necessarily always displayed in a stable manner.

In simulations performed by the inventors, however, even though there was a case where it was temporarily difficult to view a part of the contour part 42, the contour part 42 was generally always expressed. Thus, the position of the forceps 22 was able to be always recognized. Note that the specific example of the simulations will be described later by using a simulation result (FIG. 8) when only the handle 22B is removed.

Subsequently, FIG. 7 and FIG. 8 illustrate a state where only the image of the handle 22B in the forceps 22 has been removed. In the example of FIG. 7 and FIG. 8, the obstructing object removal process is performed only on the handle 22B, and the contour part 42 (the position indication image) is expressed only for the handle 22B.

When only the handle 22B of the forceps 22 is removed, the only change from the case where the entire forceps 22 is removed is that the obstructing object to be removed is the handle 22B instead of the entire forceps 22, and the obstructing object removal process can be performed in substantially the same manner in the case for the entire forceps 22.

FIG. 8 illustrates an image obtained by cutting it out from a simulation moving image of a surgery at a certain timing when the surgery is performed with the surgery support system 10 of the present embodiment. In the simulation, a model of the organ 24 is used, and the model of the organ 24 is arranged in a model of a rib 46. What is illustrated in FIG. 8 is the synthesized image 34, and the forceps 22 appears in the foreground of the organ 24 in the synthesized image 34.

In the image of the forceps 22, the pinch part 22A remains, and the handle 22B has been removed. The contour part 42 of the handle 22B surrounds the periphery of the hidden-part image 40. The synthesized image as seen in the example of FIG. 8 is defined as one frame, the image processing unit 14 sequentially outputs synthesized images for every several milliseconds to ten and several milliseconds to the display unit 16, for example, and thereby a moving image is generated. The surgical operator is able to perform the surgery on the organ 24 or the like while viewing the moving image.

In the synthesized image 34 of FIG. 8, even though there is a certain degree of mismatch between images, such degree of mismatch does not matter in performing the surgery while viewing the position or the motion of the forceps 22. Further, although an image of the whole or a part of the handle 22B may be displayed for some captured objects in the captured images 32-1 to 32-3 from the image capturing units 2-1 to 2-3, a situation where such an object is continuously displayed over time such as, for example, 1 second or longer does not occur, and the duration of display of such an image of the whole or a part of the handle 22B is only a degree that is a moment. Thus, also in this regard, this does not matter with surgical operations.

As illustrated in FIG. 7 and FIG. 8, by performing the obstructing object removal process to remove an obstructing object (the handle 22B in this example), it is possible to perform a surgery without the field of view being obstructed by the obstructing object and provide the surgery support system 10 that can ensure a better field of view.

It is possible to allow the process of removing the entire forceps 22 (FIG. 4 to FIG. 6) and the process of removing only the handle 22B (FIG. 7 and FIG. 8) to be arbitrarily selected and performed. Further, it is also possible to allow only any one of these processes to be performed. For example, it is also possible to eliminate the function of performing the process of removing the entire forceps 22 and provide only the function of performing the process of removing only the handle 22B.

Since such surgeries that use the surgical assistance device 1 are often performed in a narrow range or a dark range, it is effective in the surgeries to generate an image from which an obstructing object such as the entire forceps 22 (or the handle 22B) has been removed. Moreover, if it is not possible to determine the position of the forceps 22 at all, contact of the forceps 22 with an unexpected position may occur. Thus, it is also effective in surgeries to make it possible to determine the position of the forceps 22 even to some degrees.

Moreover, by making it possible to recognize the view of the treatment part (the pinch part 22A in this example) performing treatment, it is possible to more accurately manipulate the treatment tool (the forceps 22 in this example). Therefore, in surgeries, it is also effective to leave the treatment part (the pinch part 22A in this example) in the image.

Further, a function of enabling removal of the contour part 42 in accordance with the situation may be provided. By doing so, it is possible to perform the obstructing object removal more suited to the surgical operator's need.

Note that, when the forceps 22 moves to a corner of the field of view in each of the image capturing units 2-1 to 2-3, an image of the organ 24 or the like may be unable to be used as the hidden-part image 40. In the treatment by the pinch part 22A of the forceps 22, however, the surgical assistance device 1 is typically directed to the targeted site of the surgery. Thus, in the treatment by the pinch part 22A, it is considered that the image of the organ 24 or the like are obtained by at least any of the image capturing units 2-1 to 2-3, and problems in a surgical operation are less likely to occur.

### Autofocus Function

The surgery support system 10 of the present embodiment is provided with an autofocus function. The autofocus function is performed by the autofocus unit 74 (FIG. 3) that can adjust the focal distances of the image capturing units 2-1 to 2-3 individually. The autofocus function intends to eliminate a mismatch related to focusing.

While the image capturing units 2-1 to 2-3 have been calibrated in advance before used, the image may be out of focus and blurred (so called out-of-focus may occur), because the distance between the surgical assistance device 1 and the organ 24 or the like, which are observation targets (also referred to as “subject”), changes during a surgery.

For example, it is assumed that the focal position in calibration is at the center 44B out of three portions of the front 44A, the center 44B, and the back 44C illustrated on the left side in FIG. 9. In such a case, when the position of the organ 24 or the like moves to the front 44A or the back 44C due to a change of the position of the surgical assistance device 1, out-of-focus occurs causing the synthesized image 34 to be blurred, as illustrated in the upper part or the lower part on the right side in FIG. 9.

To prevent such out-of-focus by image processing and obtain a clear image, automatic focus adjustment via the autofocus function is performed. The autofocus function may be one relying on optical zooming or may be one relying on digital zooming. For the autofocus function, it is possible to employ one that fixes the positions of the image capturing units 2-1 to 2-3 and changes the relative distances between the image capturing units 2-1 to 2-3 by image processing to adjust the focus.

The relative distances related to the image capturing units 2-1 to 2-3 can be changed by changing the three-dimensional positions of the image capturing units 2-1 to 2-3 recognized by the image processing unit 14. The three-dimensional positions of the image capturing units 2-1 to 2-3 are relative positions based on any one of the image capturing units 2-1 to 2-3 as a reference point.

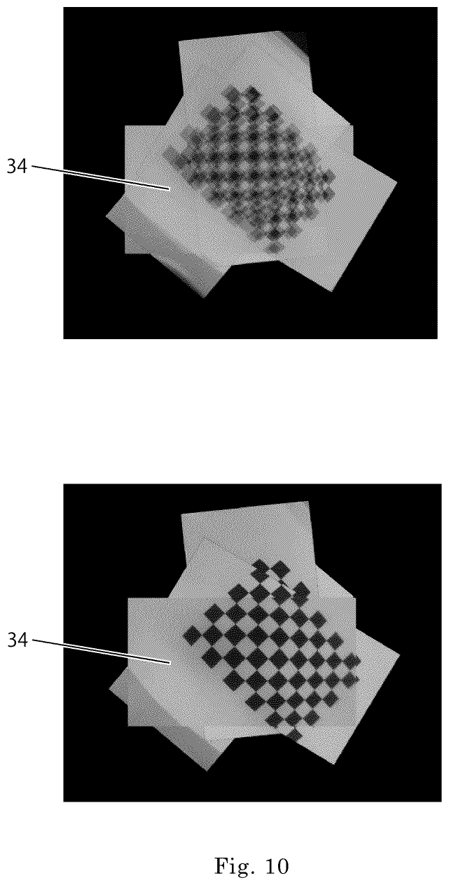

For example, a plurality of (for example, about 30) images of the calibration charts as illustrated in the synthesized image 34 of FIGS. 10(a) and (b) are captured from various directions, and thereby the relative positions between the image capturing units 2-1 to 2-3 have been estimated in advance. The estimation of the relative positions is performed by detecting corners of a rectangular in a chart and estimating a transformation (projective transformation) matrix with which corresponding positions of points of corners (corner points) captured by respective image capturing units 2-1 to 2-3 match to the detected corners. The calibration chart is held by a holder (not illustrated), and the position or the attitude (orientation) of the surgical assistance device 1 to which respective image capturing units 2-1 to 2-3 are attached is changed. Note that the positions of respective image capturing units 2-1 to 2-3 may be fixed, and the position or the attitude (orientation) of the holder of the calibration chart may be changed. A change (blur of an image) occurring when the distance from the image capturing units 2-1 to 2-3 to the subject becomes shorter or longer is apparently the same as a change occurring when the relative distances between the image capturing units 2-1 to 2-3 is increased or reduced. Thus, the edge intensity within a synthesized image is monitored while the relative distances between the image capturing units 2-1 to 2-3 is changed by parallel displacement of the image. The position at which the edge intensity within this synthesized image is maximum is found, and the blur of the image is eliminated.

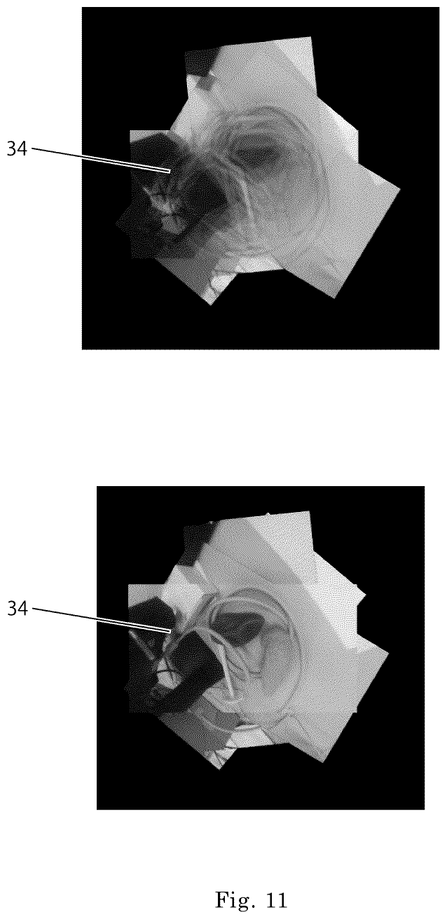

FIG. 11(a) illustrates an example of a synthesized image before autofocusing, and FIG. 11(b) illustrates an example of a synthesized image after autofocusing. What appears in FIG. 11(a), (b) is a conductive cable or the like disorderly placed on a desk, which are not an image during a surgery. However, it is found that the blurred synthesized image of FIG. 11(a) is made clear as seen in FIG. 11(b) through the autofocus function.

Herein, when the base member 4 of the surgical assistance device 1 is formed of a flexible material, the mutual positional relationship between the image capturing units 2-1 to 2-3 is likely to change during a surgery. In such a case, since the accuracy of the synthesized image 34 is likely to decrease, the autofocus function is much more effective.

Note that the autofocus function may be one that changes respective positional relationships between the image capturing units 2-1 to 2-3 separately.

### Depth Estimation Function

The surgery support system 10 of the present embodiment can be provided with a depth estimation function. The depth estimation function is performed by the depth estimation unit 76 (FIG. 3) that can estimate the depth to a point of interest inside the body cavity 20. With the depth estimation function being provided, it is possible to estimate the distance from the surgical assistance device 1 to the observation target such as the organ 24. Further, by manipulating the forceps 22 or the like while checking the estimated distance, it is possible to more reliably prevent occurrence of a situation where the forceps 22 or the like unexpectedly interfere with the organ 24 or the like.

For a method of estimating the depth, various known technologies can be employed. As a method of estimating the depth, there is a method using a geometrical solution related to cameras (the image capturing units 2-1 to 2-3 in this example), for example. In this method, the depth to the point of interest can be estimated provided that the relative positions between a plurality of cameras and the positions of the point of interest (observation target site) in the subject in respective images captured by these cameras are known.

Other than the above, distance map estimation by Cycle-GAN can be employed. The Cycle-GAN is one of the schemes to perform style conversion in a generative adversarial network (GAN) by using artificial intelligence (AI). In the GAN, the generator generates an image similar to an image of training data, and the discriminator determines whether the image is the image of the training data or the image generated by the generator. Learning is performed through repetition of this process. The style conversion is a scheme to perform conversion of the external feature of data.

Furthermore, as other methods, a method of clicking a point of interest (observation target site) in a panoramic vision and applying autofocusing to the point of interest can be employed as a method of estimating the depth.

By combining the depth estimation function employing such a method of estimating the depth with the function of the obstructing object removal process, it is possible to further enhance the safety of surgeries. Further, for example, it is possible to issue an alert via the display unit 16, a speaker (not illustrated), or the like to call attention to the surgical operator in the surgery support system 10 when the result of depth estimation indicates that a blood vessel having a predetermined thickness or greater comes to a range within a predetermined distance from the point of interest.

### 3D Measurement Function

The surgery support system 10 of the present embodiment can be provided with a function of 3D (three dimension) measurement. The function of 3D measurement intends to make a visible region viewed in a 3D manner and uses depth information on each point of the visible region. For the depth information, depth information acquired by the depth estimation can be used.

The 3D measurement enables three-dimensional surface measurement of the organ 24 or the like. For example, it is possible to find the Euclidean distance between two specified points to measure the shortest distance along the organ surface. Further, a combination of such distance measurement enables surface area measurement of the organ 24 or the like. Such a function of 3D measurement is particularly effective in incision operations or the like.

### Overall Advantage of Surgery Support System 10

According to the surgery support system 10 as described above, a better field of view can be ensured during an endoscopic surgery. Although a better field of view can be ensured even with only the obstructing object removal process (FIG. 4 to FIG. 8), much better field of view can be ensured with the autofocus function (FIG. 9 to FIG. 11) being provided. Further, a surgery can be much more accurately performed with both or any one of the depth estimation function and the 3D measurement function being provided.

### INDUSTRIAL APPLICABILITY

The surgery support system and the surgery support device disclosed in the present application can ensure a wide field of view during an endoscopic surgery, which are therefore useful for manufacturing industries for medical devices.

### LIST OF REFERENCE SYMBOLS

1 surgical assistance device
2, 2-1 to 2-3 image capturing unit
3 holding part
4 base member
10 surgery support system
14 image processing unit
16 display unit
18 operating unit
20 body cavity
22 forceps
22A pinch part
22B handle
24 organ
26, 28 peripheral object
30 synthesized image
32-1 to 32-3 captured image
34 synthesized image
36 obstructing object removed region
38 obstructing object removed image
40 hidden-part image
42 contour part
46 rib
62 control unit
64 storage unit
66 communication unit
72 obstructing object removal unit
74 autofocus unit
76 depth estimation unit
78 3D measurement unit

What is claimed is:

1. A surgery support system comprising:
a surgical assistance device configured to assist use of a treatment tool inserted into a body cavity and having image capturing units configured to capture images inside the body cavity;
an image processing unit configured to process images obtained from the image capturing units; and
a display unit configured to display image data image-processed by the image processing unit,
wherein the image capturing units are configured such that
a plurality of image capturing units are provided to the surgical assistance device,
each of the image capturing units captures an image so that inside of the body cavity appears in the image, and
when the treatment tool is inserted in the body cavity, any of the image capturing units captures an image so that the inside of the body cavity including the treatment tool appears in the image, and
wherein the image processing unit is configured to
synthesize images obtained from respective image capturing units to generate a synthesized image, and
perform an obstructing object removal process of removing at least a part of an image of the treatment tool.

2. The surgery support system according to claim 1,
wherein the treatment tool has
a treatment part used for treatment inside the body cavity, and
a non-treatment part continuous to the treatment part, and
wherein the image processing unit generates an obstructing object removed image by combining at least
a position indication image indicating a position of the non-treatment part based on the shape of the non-treatment part, and
a hidden-part image that is an image hidden by the non-treatment part.

3. The surgery support system according to claim 2, wherein the image processing unit generates the position indication image by performing machine learning on the non-treatment part from training images of the treatment tool.

4. The surgery support system according to claim 3, wherein the position indication image is an image indicating a contour of the non-treatment part.

5. The surgery support system according to claim 1 further comprising an autofocus unit configured to adjust focal distances of the image capturing units individually.

6. The surgery support system according to claim 1 further comprising a depth estimation unit configured to estimate a depth to a point of interest inside the body cavity.

7. A surgery support device comprising: an image processing unit configured to process images obtained from a plurality of image capturing units of a surgical assistance device configured to assist use of a treatment tool inserted into a body cavity,
wherein the surgery support device
synthesizes the images obtained from respective image capturing units to generate a synthesized image, and
performs an obstructing object removal process of removing at least a part of an image of the treatment tool.

8. A program used in the surgery support system according to claim 1.

9. The surgery support system according to claim 1,
wherein the surgical assistance device includes holding parts and a base member, the holding parts holding the image capturing units, respectively,
wherein the base member is formed in a tube-like shape, and the image capturing units are attached to the base member through the holding parts,
wherein three or more of the image capturing units are provided, and each of the image capturing units is arranged so as to be directed to outside of the base member at an angle of 0 degree to 10 degrees, and
wherein each of the holding parts is formed so as to penetrate through the base member.

10. The surgery support system according to claim 1,
wherein the treatment tool has
a treatment part used for treatment inside the body cavity, and
a non-treatment part continuous to the treatment part, and
wherein the image processing unit is configured to select and perform any of
an obstructing object removal process of removing the entire treatment tool, and
an obstructing object removal process of removing only the non-treatment part.

11. The surgery support device according to claim 7,
wherein the surgical assistance device includes holding parts and a base member, the holding parts holding the image capturing units, respectively,
wherein the base member is formed in a tube-like shape, and the image capturing units are attached to the base member through the holding parts,
wherein three or more of the image capturing units are provided, and each of the image capturing units is arranged so as to be directed to outside of the base member at an angle of 0 degree to 10 degrees, and
wherein each of the holding parts is formed so as to penetrate through the base member.

12. The surgery support device according to claim 7,
wherein the treatment tool has
a treatment part used for treatment inside the body cavity, and
a non-treatment part continuous to the treatment part, and
wherein the image processing unit is configured to select and perform any of
an obstructing object removal process of removing the entire treatment tool, and
an obstructing object removal process of removing only the non-treatment part.

13. The surgery support system according to claim 2 further comprising an autofocus unit configured to adjust focal distances of the image capturing units individually.

14. The surgery support system according to claim 3 further comprising an autofocus unit configured to adjust focal distances of the image capturing units individually.

15. The surgery support system according to claim 4 further comprising an autofocus unit configured to adjust focal distances of the image capturing units individually.

16. The surgery support system according to claim 2 further comprising a depth estimation unit configured to estimate a depth to a point of interest inside the body cavity.

17. The surgery support system according to claim 3 further comprising a depth estimation unit configured to estimate a depth to a point of interest inside the body cavity.

18. The surgery support system according to claim 4 further comprising a depth estimation unit configured to estimate a depth to a point of interest inside the body cavity.

19. A program used in the surgery support system according to claim 2.

20. A program used in the surgery support device according to claim 7.
